(19) United States
(12) Patent Application Publication
(10) Pub. No.: US 2019/0113529 A1
(43) Pub. Date: Apr. 18, 2019

(54) Blood Quality Diagnosis Kit

(71) Applicant: PRT LTD., Gibraltar (G) (GB)

(72) Inventor: Shumei Chen, Nanjing (CN)

(21) Appl. No.: 16/090,830

(22) PCT Filed: Apr. 5, 2016
(86) PCT No.: PCT/IB2016/051919

§ 371 Date: Oct. 3, 2018

(51) Int. Cl. G01N 33/80; G01N 33/50; G01N 33/555

(52) U.S. Cl. CPC G01N 33/80; A61M 1/0272; G01N 33/555; G01N 33/5091

(57) ABSTRACT

A diagnostic biological array, for determining quantitative and measurable blood quality and/or maximum storage period of a blood unit at the day of donation and/or survivability period (after transfusion) of 75% of the RBCs of an individual blood unit and/or comparing a blood unit quality level to the recipient blood quality prior to transfusion and/or detecting impact on blood flow and correlation to specific diseases, by using parameters which are correlated to the red blood cell flow properties (FP).

### FIELD OF THE INVENTION

The present invention generally relates to diagnosis kits and specifically to a kit for diagnosing/determining blood quality.

### BACKGROUND

Blood tests are a common practice for determining the clinical condition of a patient. The blood tests often include Red Blood Cells (“RBC”) counts, hemoglobin levels and others. However, none of the tests directly tests the RBC condition. Furthermore, RBC condition is not tested before transfusing a unit of blood to a patient.

The quality of blood and its derived applications is influenced by the cells that have low Flow Properties (“FP”). The percentages of low FP in the RBC population has to be low and we have gained the experience in setting the threshold of low FP RBC and their effect on the whole population. Thus, the FP can be characterized by certain measurable characterizations and behavior of the (virtually) individual RBC that impacts RBC Flow Properties (FP) including their elasticity/flexibility, adhesion and aggregation. In addition, the FP determines the survivability rate and the FP deteriorates over time in a predictive and measurable way. Therefore, the current FP (which is very different among people/donors) determines the effective (maximal) storage duration (that still yields 75% survivability), thus indicating the maximum storage period of blood units prior to transfusion, the RBC survivability rate in a recipient body and other quantitative and measurable indicative results of the blood. In addition, the FP of the donated blood should be at least as good as the FP of the recipient, otherwise it can cause more harm than benefit to the blood circulation of the recipient, especially in the periphery (microcirculation),

### SUMMARY

According to a first aspect of the present invention there are provided kits and methods that diagnose blood quality for certain applications.

According to a second aspect of the present invention there is provided a diagnostic biological array, for determining quantitative and measurable blood quality by using parameters which are correlated to the red cell flow properties (FP).

The parameters may comprise red blood cell (RBC) deformability, RBC aggregability and/or RBC adherence to endothelium.

According to another aspect of the present invention there is provided a diagnostic biological array, for determining maximum storage period of a blood unit on the day of donation by using parameters which are correlated to the RBC flow property (FP).

The parameters may comprise RBC deformability, RBC aggregability and/or RBC adherence to endothelium.

According to another aspect of the present invention there is provided a diagnostic biological array, for determining the survivability (after transfusion) of 75% of the RBCs of an individual blood unit by using parameters which are correlated to the RBC flow property (FP).

The parameters may comprise RBC deformability, RBC aggregability and/or RBC adherence to endothelium.

According to another aspect of the present invention there is provided a diagnostic biological array, for comparing a blood unit quality level to the recipient blood quality prior to transfusion by using parameters which are correlated to the RBC flow property (FP).

The parameters may comprise RBC deformability, RBC aggregability and/or RBC adherence to endothelium.

According to another aspect of the present invention there is provided a diagnostic biological array, for detecting impact on blood flow and correlation to specific diseases by using antibodies which are correlated to parameters which are correlated to the RBC flow property (FP).

The parameters may comprise RBC deformability, RBC aggregability and/or RBC adherence to endothelium.

The diagnostic biological array may use one or more of any combination of the following biomarkers: Band3, Band4.1, Band4.2, Adducin, Ankyrin, Actin, Spectrin, a-globin, b-globin, Annexin-V, CC1, CC3, CC4, Factor-B, C1 inhibitor, Clusterin 58 and Apolipoprotein A.

### BRIEF DESCRIPTION OF THE DRAWINGS

For better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 shows schematically RBC deformability under shear stress;

FIG. 2 shows schematically impaired blood flow pattern adhesion.

FIG. 3 shows RBC storage period in days as aggregate size increases during storage

FIG. 4A shows RBC survivability in different levels of flow property;

FIG. 4B shows the deterioration in FP of the RBC over time and deterioration in FP over time; and

FIG. 5 is a schematic representation of the workflow for using a protein chip.

### DETAILED DESCRIPTION OF PREFERRED EMBODIMENTS

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

The present invention provides a sensitive and reliable method and kit comprising a novel diagnostic biological array, for diagnosing blood quality by testing measurable Red Blood Cells (RBC) characteristics and their flow level.

Thus, the present invention provides a method for determining blood quality in a blood sample, the method comprising:

a. Obtaining a whole blood sample;

b. Lysing the cells and extracting the proteins;

c. Subjecting the lysates with the selected antibodies; and

d. determining the blood quality.

The present invention enables to predict the survivability percentage of RBC within the 24 hours following blood transfusion, the maximum storage period of a blood unit after donation (that will assure, for example, the survivability of at least 75% of the transfused RBC within 24 hours). Furthermore, the present invention provides improvement in blood flow (especially in the periphery) in people receiving blood transfusion by monitoring the transfused blood's RBC Flow Property (FP). Furthermore, the present invention provides improvement in detecting diseases and others.

The RBC characteristics and their FP level can be determined by calculating deformability and/or self aggregability and/or adhesion parameters of the individual RBC and the RBC population.

The deformability and/or self aggregability and/or the adhesion parameters of the RBC were found to determine the RBC quality and survivability.

The deformability for example can be calculated by using the Elongation Ratio (ER) of the individual RBC. ER=Elongation Ratio=major/minor axis of RBC=The change in the cell axial ratio as a function of shear stress.

ER distribution in RBC population=% of rigid, undeformable cells in RBC population.

The shape change of individual RBC in the cell population is determined (at 37° C.) with RBC attached to a slide, as a function of flow-induced shear stress. The image analysis of the cell shape, changing under shear stress (3.0 Pa), provides the elongation index for each cell, and the distribution of that index in the RBC population.

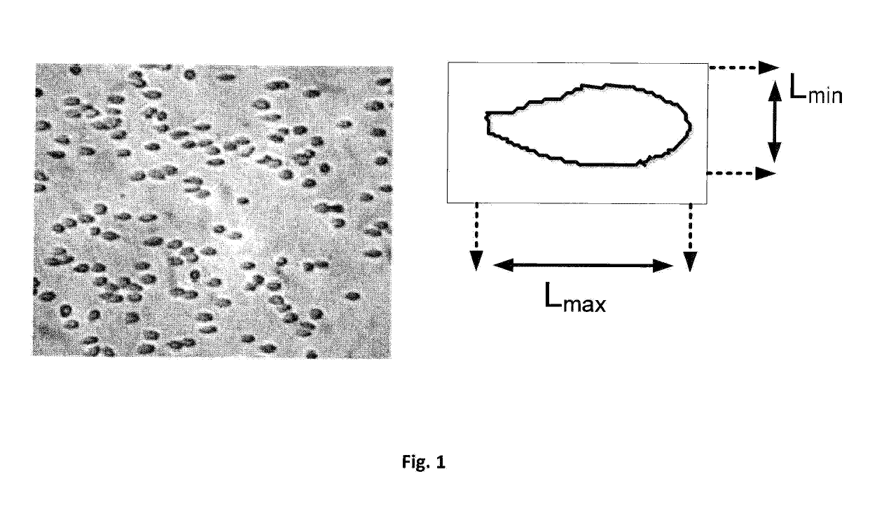

RBC deformability under shear stress (FIG. 1):

ER=L max/L min

The adhesion can be calculated for example by using the percent of RBC with Phosphatidylserine (PS) translocated to the cell surface or the percent of RBC that is attached to endothelial cells (EC). The PS externalization can be determined by Fluorescence-activated cell sorting (FACS) of intact cells using a PS-specific ligand. Surface PS is a mediator of RBC/EC adhesion and its level increases with RBC aging and storage duration.

Parameters of RBC adherence to endothelium:

N=N∞+α/τ

### Where:

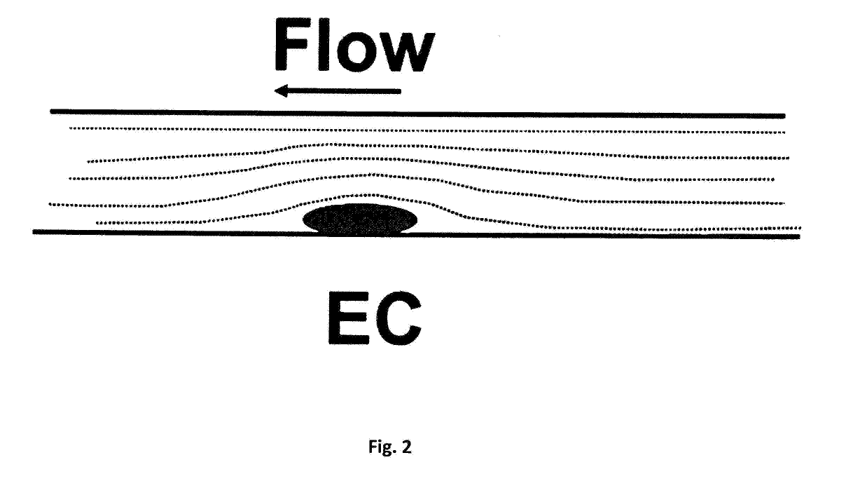

N=Number of RBC adhered to EC (or an adhesive substratum) at discrete shear stress, τ.
N∞=Number/% of “undetachable” RBC (do not detach at high τ)
α=adherence coefficient=the intercellular interaction strength.
Adhesion leads to an impaired flow pattern (FIG. 2).
Determining RBC adhesion to EC:

Δ=0.5P×H/Z

### Where:

Δ=Wall shear stress
P=Pressure at the entrence to the flow-chamber
Z=Chamber length

### H=Chamber gap

Parameters of RBC Aggregability:

ASD=Aggregate Size Distribution of RBC population into aggregate size (number of cells/aggregate) vs. Shear Stress.

AAS=Average Aggregate Size=number of RBC/number of aggregates.

SAF, LAF=Small and Large Aggregate Fractions, corresponding to size ranges of 1-4, 5-32 or >32 RBC/aggregate, respectively.

ASP=Aggregate Shape Parameter=ASP=4πA/P2, where A is the projected area of the aggregate and P is its perimeter.

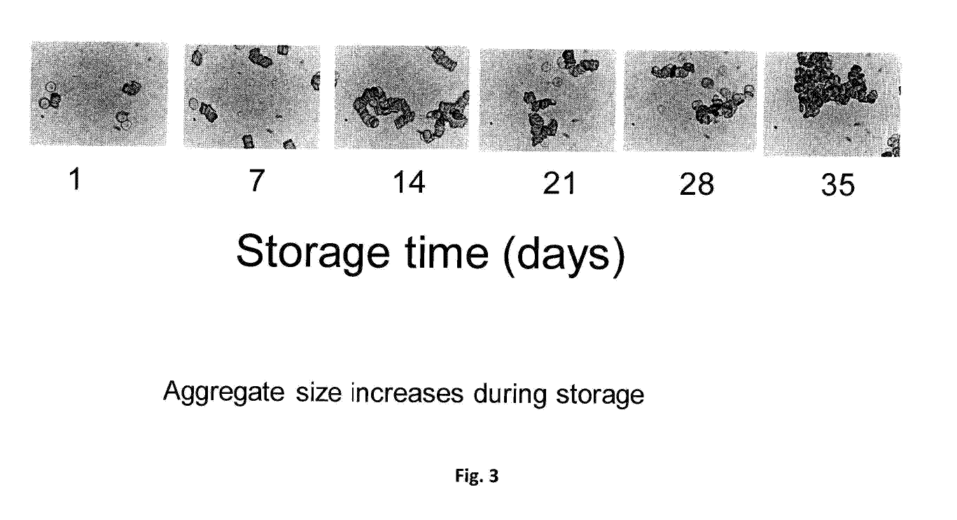

FIG. 3 shows shows RBC storage period in days as aggregate size increases during storage

The above parameters comprise the RBC FP.

These parameters were found as correlated to the survivability percentage of RBC within the 24 hours after transfusion, maximum blood unit storage after donation, blood flow, detection of diseases and others.

We have taken RBC with different FP and analyzed the expression levels of the different proteins (by mass-spectrometry) in these samples.

The proteins that show significant difference are listed below:

Band3, Band4.1, Band4.2, Adducin, Ankyrin, Actin, Spectrin, a-globin, b-globin, Annexin-V, CC1, CC3, CC4, Factor-B, C1 inhibitor, Clusterin 58, Apolipoprotein A.

The results of each test are derived by receiving the intensity of the signal of the antibody which correlates to the concentration of its target (the biomarker).

The invention enables to produce a KIT array with methods and parameters that enable relatively faster and further accurate results in blood quality detection.

### The Kit Algorithm and Process:

The antibodies array chip is a glass slide with antibodies that are attached to it. The antibodies correspond to the biomarkers.

When the sample (RBC lysate) is placed on the chip, the proteins of interest bind to their corresponding antibodies.

A good wash of the slide will clear away the other (irrelevant) proteins from the slide.

Another set of antibodies, correspond to the proteins of interest (but for another epitope) is used for higher specificity.

The antibodies are conjugated to a fluorophore that can be read by a florescent reader.

When a protein is expressed more in the sample, more antibodies (specific) will bind to it and will induce a higher signal.

The signal is converted to numbers by the computer that works with the reader.

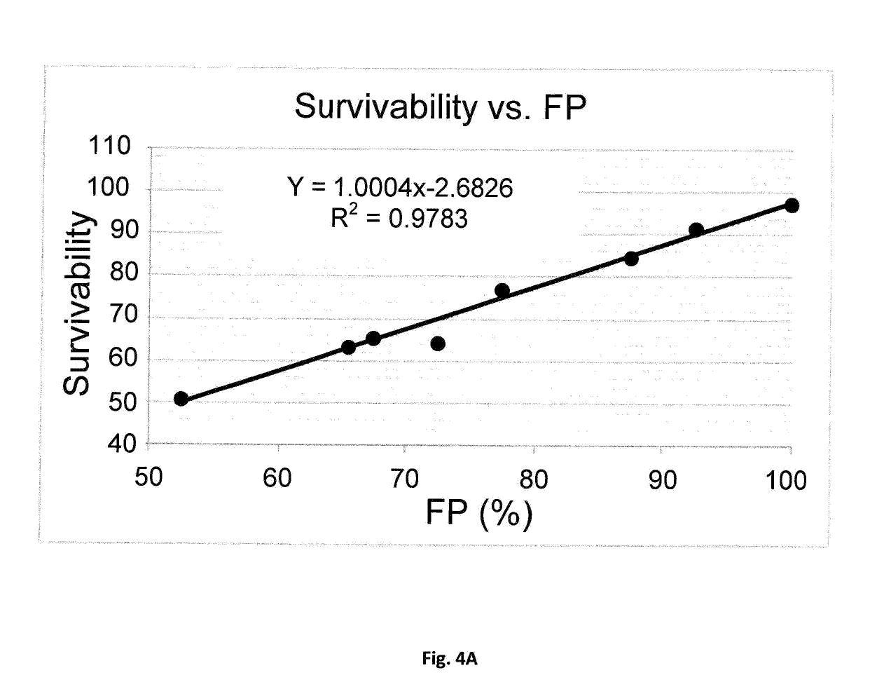

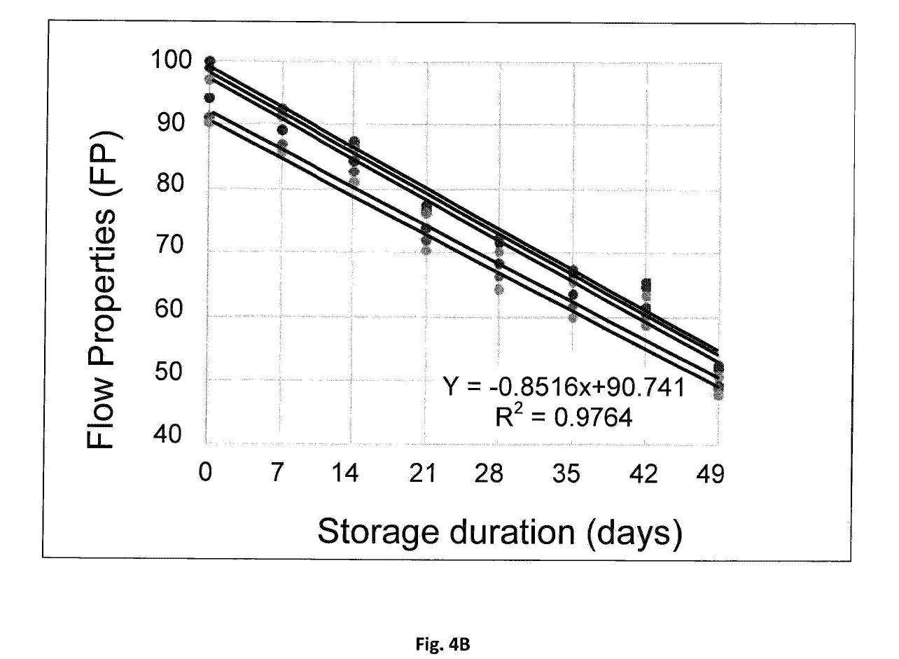

Based on trials, a survivability curve (FIG. 4A) between the expression level of the proteins and the RBC survival in the recipient may be constructed, that serves as a basis for an algorithm that converts the readers' parameters directly to survivability.

The same may be done with storage duration (FIG. 4B).

Taking the two (survivability & storage) graphs (equations) together we are able to determine directly for how long a blood unit can be stored and will not fail to yield 75% survivability within the 24 hours after transfusion.

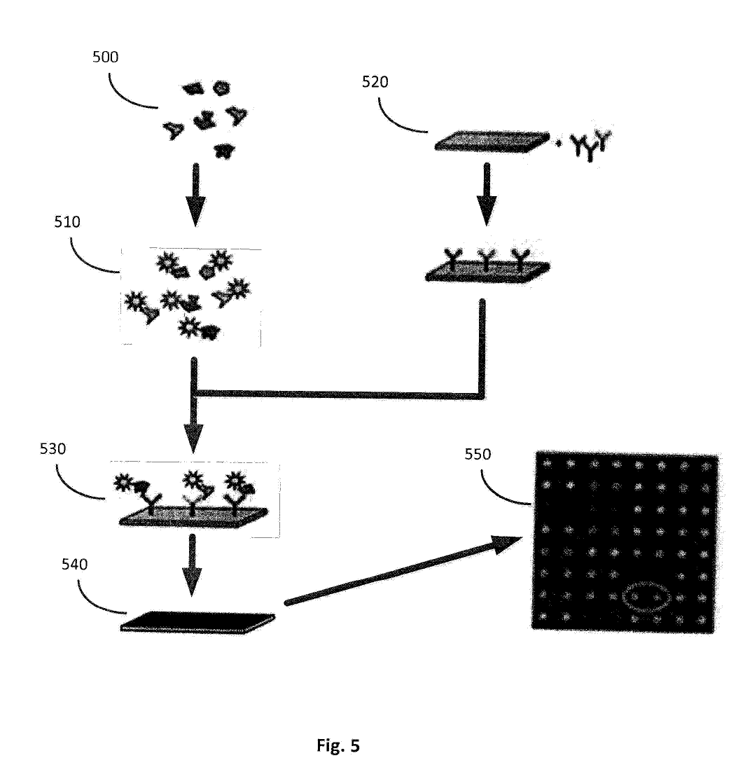

FIG. 5 is a schematic representation of the workflow for using the antibodies array and method of the present invention.

Step 500: Protein extraction from the blood sample.

Step 510: Direct sample labeling with fluorescent dye.

Step 520: Antibody immobilization on array surface.

Step 530: Sample incubation on antibody array.

Step 540: Image acquisition with fluorescence scanner.

Step 550: Spot quantification and data analysis.

It will be appreciated by persons skilled in the art that the present invention is not limited to what has been particularly shown and described hereinabove. Rather the scope of the present invention is defined by the appended claims and includes combinations and sub-combinations of the various features described hereinabove as well as variations and modifications thereof which would occur to persons skilled in the art upon reading the foregoing description.

1. A diagnostic biological array chip, for determining quantitative and measurable blood quality, configured to carry antibodies selected to correspond to selected biomarkers for measuring parameters which are correlated to the red blood cell (RBC) flow properties (FP).

2. The diagnostic biological array chip of claim 1, wherein said parameters comprise RBC deformability.

3. The diagnostic biological array chip of claim 1, wherein said parameters comprise RBC aggregability.

4. The diagnostic biological array chip of claim 1, wherein said parameters comprise RBC adherence to endothelium.

5. A diagnostic biological array chip, for determining maximum storage period of a blood unit on the day of donation, configured to carry antibodies selected to correspond to selected biomarkers for measuring parameters which are correlated to the RBC flow property (FP).

6. The diagnostic biological array chip of claim 5, wherein said parameters comprise RBC deformability.

7. The diagnostic biological array chip of claim 5, wherein said parameters comprise RBC aggregability.

8. The diagnostic biological array chip of claim 5, wherein said parameters comprise RBC adherence to endothelium.

9. A diagnostic biological array chip, for determining the survivability (after transfusion) of 75% of the RBCs of an individual blood unit, configured to carry antibodies selected to correspond to selected biomarkers for measuring parameters which are correlated to the RBC flow property (FP).

10. The diagnostic biological array chip of claim 9, wherein said parameters comprise RBC deformability.

11. The diagnostic biological array chip of claim 9, wherein said parameters comprise RBC aggregability.

12. The diagnostic biological array chip of claim 9, wherein said parameters comprise RBC adherence to endothelium.

13. A diagnostic biological array chip, for comparing a blood unit quality level to the recipient blood quality prior to transfusion, configured to carry antibodies selected to correspond to selected biomarkers for measuring parameters which are correlated to the RBC flow property (FP).

14. The diagnostic biological array chip of claim 13, wherein said parameters comprise RBC deformability.

15. The diagnostic biological array chip of claim 13, wherein said parameters comprise RBC aggregability.

16. The diagnostic biological array chip of claim 13, wherein said parameters comprise RBC adherence to endothelium.

17. A diagnostic biological array chip, for detecting impact on blood flow and correlation to specific diseases, configured to carry antibodies selected to correspond to selected biomarkers for measuring parameters which are correlated to the RBC flow property (FP).

18. The diagnostic biological array chip of claim 17, wherein said parameters comprise RBC deformability.

19. The diagnostic biological array chip of claim 17, wherein said parameters comprise RBC aggregability.

20. The diagnostic biological array chip of claim 17, wherein said parameters comprise RBC adherence to endothelium.

21. The diagnostic biological array chip according to claim 1, using one or more of any combination of any of any biomarkers biomarkers including:
Band3, Band4.1, Band4.2, Adducin, Ankyrin, Actin, Spectrin, a-globin, b-globin, Annexin-V, CC1, CC3, CC4, Factor-B, C1 inhibitor, Clusterin 58 and Apolipoprotein A.

22. A method of measuring blood parameters pertaining to predefined proteins or biomarkers of interest, comprising:
obtaining a blood sample;
extracting proteins from the blood sample;
labeling said extracted proteins with fluorescent dye;
attaching antibodies corresponding to said proteins or biomarkers of interest to an array chip;
incubating said labeled proteins on said array chip;
acquiring an image of said array chip with a fluorescence scanner; and
spot quantifying and analyzing said image data.
